(19) United States
(12) Patent Application Publication
(10) Pub. No.: US 2006/0110292 A1
(43) Pub. Date: May 25, 2006

(54) Systems, method and devices for monitoring fluids

(76) Inventors: Richard A. Deverse, Kailua Kona, HI (US); Van Malan, Kailua Kona, HI (US); Andreas Coppi, Groton, CT (US); William G. Fateley, Manhattan, KS (US); Frank Geshwind, Madison, CT (US)

Correspondence Address: C. Andrew Im;FULBRIGHT & JAWORSKI L.L.P., 666 Fifth Avenue, New York, NY, 10103, US

(21) Appl. No.: 11/244,596

(22) Filed: Oct. 5, 2005

Related U.S. Application Data
(60) Provisional application No. 60/616,402, filed on Oct. 5, 2004.
(60) Provisional application No. 60/619,047, filed on Oct. 15, 2004.
(60) Provisional application No. 60/624,971, filed on Nov. 3, 2004.
(60) Provisional application No. 60/712,076, filed on Aug. 29, 2005.
(60) Provisional application No. 60/712,163, filed on Aug. 29, 2005.

(51) Int. Cl. G01N 33/00

(52) U.S. Cl. 422/68.1

(57) ABSTRACT

Method and apparatus for detecting leaks in a fluid vessel. The apparatus comprises a pressure sensor for measuring a pressure difference between a reference cell and a sample cell. The sample cell is open to the fluid vessel. The method and apparatus detects a leak in the fluid vessel when the pressure difference exceeds a predetermined threshold.

### RELATED APPLICATION

This application claims priority benefit under Title 35 U.S.C. §119(e) of provisional patent application Nos. 60/616,402 filed Oct. 5, 2004, 60/619,047 filed Oct. 15, 2004, 60/624,971 filed Nov. 3, 2004, 60/712,076 filed Aug. 29, 2005, and 60/712,163 filed Aug. 29, 2005, each which is incorporated by reference in its entirety.

### BACKGROUND OF THE INVENTION

The present invention relates to environmental monitoring, more particularly to a system and method for detecting leaks and analyzing chemical constituents of a fluid.

Color changes as a result of direct and indirect chemical reactions have been developed for many years to aid the analytical chemist micro-biologist and health practitioners to measure qualitatively and quantitatively constituents of interest such as inorganic, organic and biological materials in our environment or in collected samples of material. These colorimetric methods can be automated such that instead of using the human eye to observe and assess the calorimetric properties, one can use a spectrometric system that is sensitive to the calorimetric process of interest. The system may include growth agents that work to grow the biological material of interest such as molds, fungi, virus and bacteriological species. These growth agents can contain colorimetric indicators that are indicative and specific or non-specific to a particular strain or species of organic material. This spectrometric approach allows a more precise determination of quantitative measure of the colorimetric change that is not possible with the human eye. This precise data can then be related to the quantitative measure of interest, digitized and integrated with any number of other environmental parameters to gain knowledge of the environment. In accordance with an embodiment of the present invention, the system and method can be used to biologically and chemically monitor the water supply systems.

Reagent-based colorimetric analysis of fluid samples is a standard technique for qualitative and quantitative chemical analysis with many application areas ranging from water quality analysis to biomedical analysis. There are many instantiations of the basic technique which differ in their accuracy, sensitivity, objectivity, cost of consumables, and cost of instrumentation.

The simplest method of colorimetric analysis is a reagent test strip that changes color intensities or color with the change in constituents found in the sample under test. A reagent test strip is illustrated in FIGS. 3 and 4. Blocks of reagent impregnated paper or plastic material are deposited upon a backing strip. Typically multiple blocks of reagent are deposited if it is desired to test for multiple chemical constituents. In the simplest case, each block tests for a single chemical constituent, and different blocks on the same strip test for possibly different chemical constituents. The test strip is immersed in the fluid or an extraction of the fluid to be analyzed. The reagent blocks change color in response to chemical reactions occurring between the reagent and the chemicals to be analyzed in the fluid. The changed colors are observed by the human eye and matched to a known chart of colors, with different colors corresponding to different concentrations of a given chemical constituent of the fluid to be tested.

This method is economical because no extra equipment beyond the human eye is required. Also the reagent test strips are inexpensive to manufacture, simple to use in the field, and are very portable. However subjective color comparison methods like this are known to be less reliable, require more user training for accuracy, are unusable by colorblind individuals, are sensitive to lighting conditions, are sensitive to reagent dilution variability, are sensitive to reagent bleeding from one pad to another, and are sensitive to lot-to-lot variations in the test strip manufacture.

A more objective calorimetric measurement method is to mix in a reusable cuvette a mixture of the fluid to be tested with a calorimetric reagent indicator chemical. The resulting mixture can be calorimetrically measured by a calorimeter or a spectrometer instrument. This method is not subjective and is capable of higher accuracy than the human eye. Also, the only consumable is the reagents that are mixed into the fluid to be tested. However, the measuring calorimeter or spectrometer is typically expensive. Another major problem with such a cuvette system is that the precise measurement of the reagents for mixing in the cuvette is labor intensive, requires skill, requires training for reliable reproducibility and is thus unsuitable for many applications and prone to error. Also, only one colorimetric test can be performed at a time, compared to the first method which performed as many tests as there are different reagent blocks on the test strip.

Another method for colorimetric measurement involves the use of a calorimeter or spectrometer with pre-prepared cuvettes which are manufactured with the reagents already in them. This has all the advantages of the previous method while avoiding the labor and skill required for dispensing the reagents. However, the cuvette is now a consumable item and can be relatively expensive, and there is still a reasonable amount of skill and training required to reduce errors.

There is continued interest in the development of new devices and methods for reagent-based calorimetric analysis with low cost consumables but high accuracy and objectivity. In many situations, it will be preferable if the calorimetric analysis method is capable of high throughput, performing many colorimetric tests simultaneously. In some situations, it will be preferable if the calorimetric analysis instrumentation is rugged, small, self-contained and portable so that the instrument may be brought to the fluid rather than the fluid being brought to the instrument. In some situations, it will be preferable if the colorimetric analysis instrumentation can be sealed so that it can be dipped into the fluid to be measured without damaging the analysis instrument. In some situations it is preferred to reduce the number of regent pads or cuvettes so that the ability to multiplex the calorimetric reagents would be an advantage. This is the case where multiple calorimetric reagents are present in the sample under test simultaneously such that multiple constituents may be analyzed simultaneously by an analyzer capable of such a measure. This measurement would be accomplished at a multitude of frequencies of light by a device made for such a measure.

Accordingly, it is desirable to have methods and devices for reagent-based colorimetric analysis of fluids that has at least some of the advantages described, while avoiding at least some of the disadvantages of prior art systems. For different circumstances and applications, different sets of advantages and disadvantages will be relevant, and the invention disclosed herein provides a number of embodiments to address some of these various tradeoffs.

Current pool leak detection system and method consists of using a pail or bucket to test for a leak over an extended period of time, see e.g., U.S. Pat. No. 6,532,814, U.S. Pat. No. 5,551,290, or American Leak Detection's Leaktell product. The leak test is conducted by partially filling a bucket or pail with the fluid under test and placing the pail or bucket in a filled fluid container under test. A mark is made to record the level of the fluid inside the bucket or pail at the level of the fluid inside the bucket or pail and also on the outside to record the level of the fluid in the container under test. After a period of at least 24 hours the level change of the fluid in the bucket or pail is compared to the level change of the container as recorded on the outside of the bucket or pail. The difference between the measures indicates the magnitude of the leak in the container under test. This method can also be implemented with a load cell where the difference is measured by a load cell using the Archimedes principle of displacement. Both of these have the disadvantage of low sensitivity and extended period of test.

Other systems, such as those described in U.S. Pat. Nos. 5,065,690 and 5,261,269 rely on administering a dye solution in the proximity of a suspected leak in order to verify and specifically locate the leak. However these systems are typically only used for locating larger and already detected leaks in accessible and easily observable locations. They cannot exhibit the accuracy and sensitivity of the present invention. A third system, as described in U.S. Pat. No. 5,734,096, uses a float system to accomplish the same task, probing specific locations for leaks with coarse accuracy and sensitivity.

Another product from American Leak Detection, the Leaktell 2 device, uses a laser rangefinder to measure the distance from a fixed point to a float in a chamber whose level tracks that of the pool it is immersed in. The laser rangefinder includes precision electronics to measure the minute amount of time it takes for the beam to bounce off a target and return to a detector on the device. This system, while accurate, is prohibitively expensive.

Consequently there is a need for devices, methods and systems that can monitor for leaks in pools and containers that is at least some of: faster, more accurate, more precise, more robust and less expensive than prior art systems.

Prior art sensor arrays consist of discrete sensor of the same type for a specific measure of interest. These sensor arrays have the disadvantage of providing single dimensional data that may or may not provide the information needed to assess the situation or measure of interest. Information is needed from a multitude of various sensors where each only delivers a part of the whole of the information that is needed for a proper assessment of the situation or condition of interest that is of higher dimensionality and requires a suite of sensors arranged in arrays that provide the multi-dimensional data needed for a proper analysis of a situation or condition of interest.

Consequently, there is a need for complex arrays of devices, methods and systems for monitoring and/or tracking of complex system states in arrays of different or similar sensors, that are at least some of: faster, more accurate, more precise, more robust, less expensive and dimensionally deep than prior art systems.

The term environmental monitoring is used broadly herein, to refer to any and all circumstances and conditions by which there is at least on sensor in a particular local or extended environment measuring one or more parameters of that environment. The sensor used can be a complex device such as a spectrometer or a simple transducer such as a photocell. The sensor can be a system or array of organic materials that functions to capture, grow and sustain a culture or community of biologicals for sensing by means of calorimetric media changes or other electronic or electro-optic or optical means. Some of the embodiments described will consist of arrayed colorimetric sensors as sensor sub-systems in a networked-enabled modular monitoring and information delivery system. Such arrangements are useful in many applications, including but not limited to automated monitoring for preventative maintenance of piping and other fluid delivery systems, leak detection, chemistry, bacteriology, molds and fungi monitoring.

### OBJECT AND SUMMARY OF THE INVENTION

Therefore, it is an object of the present invention to provide a device, method and system for monitoring and/or tracking of system states and chemistry equilibrium changes in a complex system.

Another object of the present invention is to provide arrayed sensors as sensor sub-systems in a networked-enabled modular monitoring and information delivery system.

A further object of the present invention is to provide a system, method and device for detecting leaks and more particularly to monitoring and measuring leaks in a swimming pool, spa or any container containing fluidic materials under static or steady state conditions. The system, method and device of the present invention allow one to make such measurements with a high degree of confidence and with simple operation.

A still further object of the present invention is to provide a system, device and method for analyzing chemical constituents of a fluid, including but not limited to pool water and human body fluids, based on calorimetric methods applied to reagents deposited or absorbed upon a test strip.

A yet another object of the present invention is to provide a system, device and method for analyzing chemical constituents of water in a sample container which provides a more accurate analysis of the chemical constituents of interest than what is currently possible with the conventional methods of using the eye as a measurement comparison tool.

A still yet another object of the present invention is to provide a system, device and method for detecting and analyzing molds, fungi and other biological systems.

In accordance with an embodiment of the present invention, the system and device comprises a water quality monitor. It is appreciated that the present invention does not preclude other similar monitoring situations that can benefit from this invention. Typically, the water quality monitoring is accomplished in the laboratory using wet chemical and other methods that can involve using sophisticated methods and equipment. In accordance with an embodiment of the present invention, the required measures are automated and integrated to provide a greater amount of information in a simpler way.

In accordance with an embodiment of the present invention, the system comprises other or additional sensors, such as acoustic sensors on pipes together with components for determining the flow through the pipes, as disclosed herein. Additional sensors in this regard can include but are not limited to sensors for water chemistry such as those disclosed herein or other water chemistry sensors, micro-biology sensors, pipe corrosion sensors, particulate measuring sensors, electrical sensors, electro-chemical sensors, pressure measuring sensors, and flow measuring sensors. In addition to sensors, the systems can comprise other components such as singular or combinations of active and or passive acoustic elements. The use of active acoustic components comprises actively transmitting encoded audio energy from the active element, and then correlating this encoded energy with the energy measured at the acoustic sensors, whereby information can be obtained about the pipes including but not limited to information about the geometry of the pipes, the condition of the pipes, the presence of fluid and/or other material within the pipes. The system can also comprise gas injection systems that are activated once an anomalous acoustic signal is detected. The anomalous acoustic signal can be determined to be a probable leak and a gas, such as nitrogen, can be automatically injected and the resultant acoustic signal analyzed.

In accordance with an embodiment of the present invention, the system comprises a micro-electro-mechanical-system (MEMS) or other differential pressure transducer with a reference cell for detecting leaks. The result is a very sensitive leak sensor that can detect very small leaks. The sensor of the present invention can be made inexpensively to be operated by unskilled users. A simple “traffic-light”-like interface: red, green and amber lights, can be used to provide information of a leaking container, non-leaking container or a non-test.

In accordance with an embodiment of the present invention, the reference cell and other cell have the same geometry and material properties, thus allowing the cancellation of cell-resonance induced reading.

In accordance with an embodiment of the present invention, the system and method performs reagent-based colorimetric analysis of fluids with a greater precision than is typically accomplished by systems relying on visual inspection of color changes. The present invention utilizes colorimetric sensing while minimizing the cost of the consumables. In accordance with an aspect of the present invention, the system and method performs reagent-based calorimetric analysis of fluids without requiring user expertise in preparing the sample or reagent. Accordingly, the present invention utilizes test strips, rather than, for example, cuvettes.

In accordance with an embodiment of the present invention, the system and method performs reagent-based calorimetric analysis of fluids in an automated way, thereby permitting the system to be placed in a fixed location to automatically or periodically perform measurements, including but not limited to a pool, or can be placed on a pole or tether. That is, the present invention automatically reads and reports the measurement without user intervention. The present invention comprises devices and methods for performing calorimetric analysis of fluid samples. In an exemplary embodiment, the device comprises one or more calorimetric sensors in one or more locations on and/or in the device. In these embodiments the device also has a channel or other method for holding a test strip with reagent blocks, and has some channel or method to guide a fluid sample to the locations of the reagent blocks. In some embodiments, the spacing and placement of the sensors correspond to the spacing and placement of the reagent blocks on the strip. For example, the interval between the sensors may be equal to the interval between the reagent blocks, or proportional in the case where, for example, concentrating optics are present in the system. In some embodiments, the test strip may be placed over the device so that the reagent blocks align with the locations where the colorimetric sensors can make measurements. The reagent block is observed by a colorimetric sensor that may or may not be specific for that particular reaction combination. Readings from each of the colorimetric sensors are transmitted to a computational device which interprets the measurements and reduces them to estimates of the chemical concentrations in the fluid.

It should be noted that different embodiments of the invention may incorporate different combinations of the foregoing, and that the invention should not be construed as limited to embodiments that include all of the different elements. Various other objects, advantages and features of the present invention will become readily apparent from the ensuing detailed description, and the novel features will be particularly pointed out in the appended claims.

### BRIEF DESCRIPTION OF THE DRAWINGS

The present invention will be understood and appreciated more fully from the following detailed description, taken in conjunction with the drawings in which:

FIG. 1 shows a fluid chemistry analysis renewable media module in accordance with an embodiment of the present invention;

FIG. 2 shows the deployment of a networked sensor array system in accordance with an embodiment of the present invention;

FIGS. 3 and 4 show a view from above of a reagent test strip and a view from the side, respectively, in accordance with an embodiment of the present invention.

FIG. 5 shows a test strip and reader configuration in accordance with the present invention;

FIG. 6 shows the calorimetric sensor operating in a transflectance mode in accordance with an embodiment of the present invention;

FIG. 7 shows the colorimetric sensor operating in a transmission mode in accordance with an embodiment of the present invention;

FIG. 8 illustrates a single colorimetric sensor multiplexed among reagent blocks in accordance with an embodiment of the present invention;

FIG. 9 illustrates a typical time evolution of signal intensity from a colorimetric sensor in accordance with an embodiment of the present invention;

FIG. 10 shows a block diagram a system and device for monitoring of leaks in pools and other containers in accordance with an embodiment of the present invention;

FIG. 11 shows a pool leak detection system in accordance with an embodiment the present invention comprising two chambers, a sensor system, a processing system, and an LED;

FIG. 12 illustrates an exemplary embodiment of an electronics module and a detection/monitoring device of the present invention;

FIG. 13 shows how a laser or other light source can be used to measure water level changes in accordance with an embodiment of the present invention;

FIG. 14 shows how a system can be arranged to measure the level change of a material where magnification of the indication of the level change is desired in accordance with an embodiment of the present invention;

FIG. 15 shows a diffractive method using a slit and coherent source to ascertain the magnitude of level changes and movement in accordance with an embodiment of the present invention;

FIG. 16 shows how direct imaging using magnifying or imaging optics and a digital camera can be used in accordance with an embodiment of the present invention to detect level change;

FIG. 17 shows how a broadband light source and a linear or 2D detector can be used in accordance with an embodiment of the present invention to detect level changes by the analysis of the pattern movement on the detector with time;

FIG. 18 illustrates the direct movement of the source beam caused by material level change in accordance with an embodiment of the present invention;

FIG. 19 shows a mirror-hinge-float assembly to detect leaks or level changes in accordance with an embodiment of the present invention;

FIG. 20 shows an empty calibration position of mirror float assembly such that the mirror is normal or at a fixed position when no material is present or under test, in accordance with an present invention; and

FIG. 21 shows how displacement on a sensor corresponds with change in fluid level in accordance with an embodiment of the present invention.

FIG. 22 shows an example of a schematic for an electronic component of one embodiment of the described pool leak detector in accordance with the present invention.

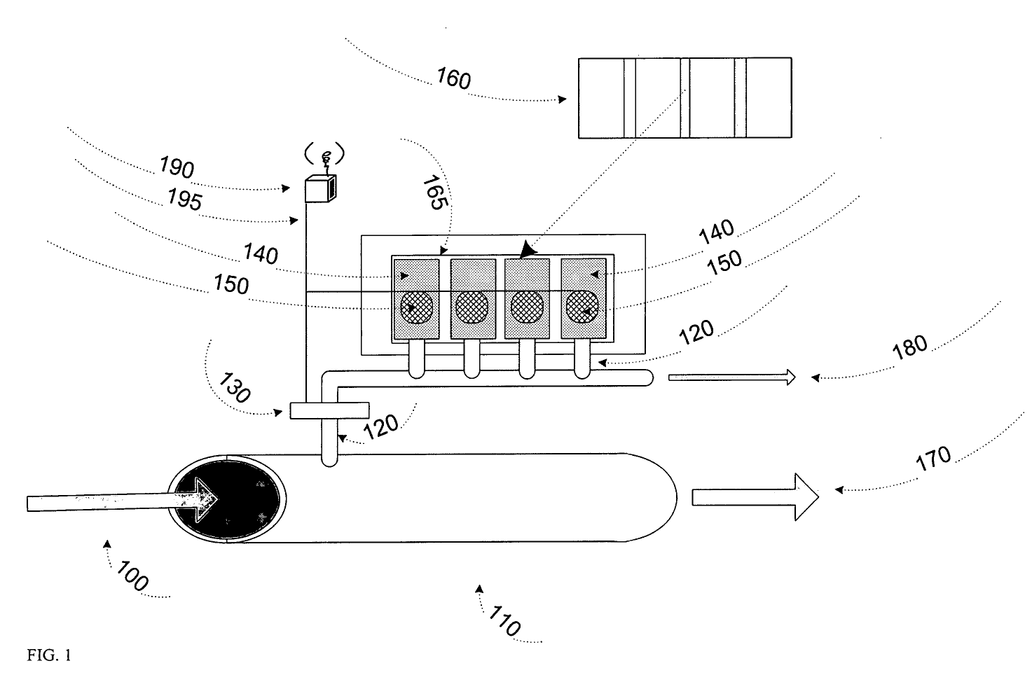

Turning now to FIG. 1, there is illustrated a cartridge of an array of calorimetric reactive reagents which is continuously inoculated by a delivery mechanism such as capillary tube for liquids. Fluid flows into an opening 100 of a tube or pipe section 110 and exits from an opening 170 of the tube or pipe section 110. An additional tube or pipe assembly 120 is coupled to the pipe section 110 so that some of the fluid will flow into the pipe assembly 120. Optionally, a flow control component 130 comprises a shut-off valve and/or a check valve. The pipe assembly 120 is disposed to deliver some fluid to one or more specific development media 140. For simplicity, it is appreciated that only two of the four development media 140 is labeled. An array of specific sensor devices 150 comprises elements sensitive to the chemistry in the specific reaction development media 140. In accordance with an embodiment of the present invention, an array or micro-array of the development media and a cartridge 160 that is accepted by a retaining mechanism 165 and automatically aligned with the sensor array system 150 when inserted. This alignment is accomplished by the retaining mechanism 165 which can comprise a set of clips and guides, and can optionally comprise a component for performing alignments, such as mechanical components for adjusting the position of the cartridge 160, mechanical and/or optical sensors for sensing the placement of the cartridge 160 and components to couple these adjusting and sensing components in a feedback loop. Waste water can optionally flow out of an opening 180 of the pipe assembly 120. A micro-controller and transmitter 190 can be coupled to the detector array 150 and optionally coupled to a flow control component 130.

In accordance with an embodiment of the present invention shown in FIG. 1, each specific colorimetric reagent spatial extent and position on the cartridge 160 is known. For each reaction, a colorimetric response is known and a spatially and spectrally matched colorimetric sensor 150 is used to measure these calorimetric reactions with time. A transmitter/micro-controller device 190 collects and encodes the signals. As shown in FIG. 1, an array of sensors is integrated into the system for each node so that the system can continuously collect more information and more knowledge of the total piping system from the structural information, such as wall corrosion in the pipe to leaks, and chemical and biological events that are monitored. A multitude of sensor signals can be integrated into the sensor array found at each node in the network of sensor array platforms. FIG. 1 also shows a replaceable reaction module or cartridge 160 in accordance with an embodiment of the present invention. It is anticipated that some reagents will only be effective for a limited time and will need to be renewed on a regular basis. Also if the reactive reagent has been used up in a reaction it can be replaced. Additionally, the present invention enables the user/operator to alter the monitoring program and select additional constituents to monitor. This can be used for thin layer chromatography (TLC), calorimetric reagents, selective or unselective media. If a reactive agent is present in the material then the colorimetric reaction will take place. Time may be an important dimension that can also be coupled to the data from the particular sensor and used to detect the time dependent presence of a contaminant and also in some cases the concentration present. For micro-biological contamination, this could be the rate at which the calorimetric growth media changes color and is indicative of species, growth cycle, inoculation event and number of bacteria present in the inoculation event. Using the present invention, one can continuously monitor the quality of potable water in the piping systems or other water supplies, create contamination warning systems, provide data to understand corrosion events, and monitor structural changes in the pipe.

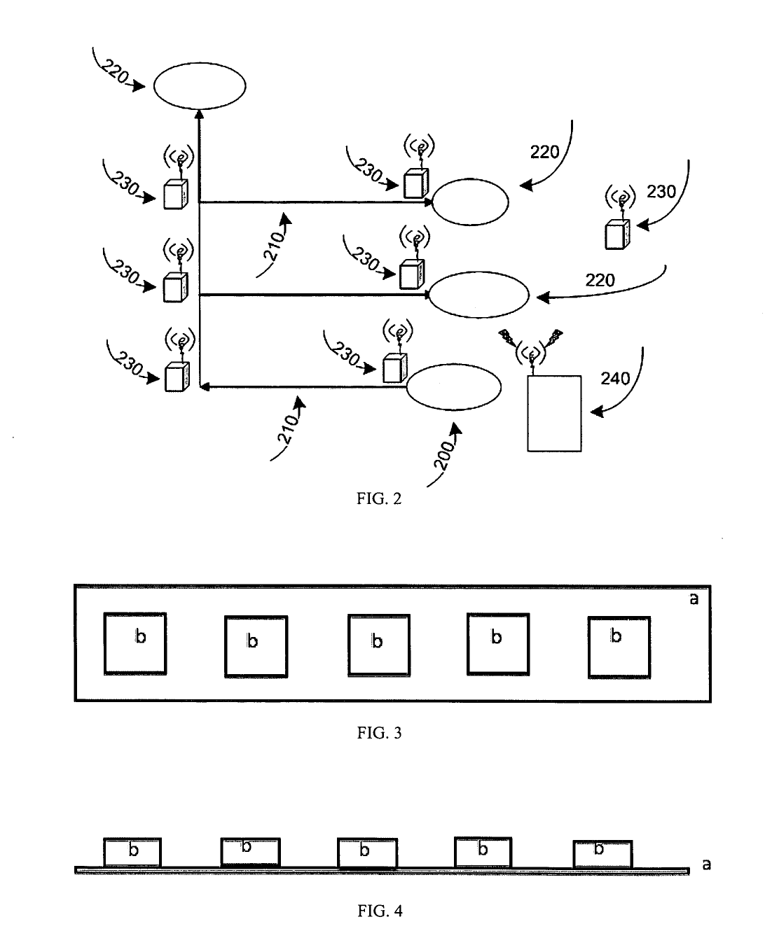

FIG. 2 shows a block diagram of a pipe health maintenance system in accordance with an embodiment of the present invention that employs methods, systems and devices disclosed herein. A water source 200 supplies water into a pipe assembly 210, which delivers the water to a plurality of water destinations 220. An array of sensor, transmission and optionally computation and processing nodes (collectively the “nodes” 230) are distributed throughout the pipe assembly 210, so that individual nodes 230 are proximal to at least some of the water destinations 220. Additionally, the nodes 230 can also be distributed at key other locations along the pipe assembly 210, including but not limited to places comprising emergency cutoff valves. The pipe health maintenance system shown in FIG. 2 in accordance with an embodiment of the present invention can be deployed within a home, apartment, other dwellings, business or residential unit, and the water destinations 220 can include, but are not limited to sinks, toilets, hot water heaters, tubs, showers, and other household plumbing fixtures.

The pipe health maintenance system shown in FIG. 2 in accordance with an embodiment of the present invention can be deployed within a municipal area, and the water destinations 220 can include but are not limited to homes, industrial buildings, public parks and hydrants. Each node 230 supports an array of sensor systems. Node sensor data is transmitted to the CPU where the data is integrated, processed and recorded. Although wireless network is shown in FIG. 2, it is appreciated that this transmission can be accomplished via wire or wireless networks of components. The wireless system can be a WiFi system, operable when the CPU's WiFi module is within range of the sensor's WiFi module(s), or it could be based on, for example, cellular network technology.

In accordance with an embodiment of the present invention, acoustic signals are measured with vibration sensors, hydrophones, microphones, and other acoustic sensors. These sensors are placed individually or in array configurations and are deployed on the inside or outside of pipes and valves in the system, or in proximity to individual pipes or groups of pipes and valves. Each sensor or sensor array is wirelessly connected in a mesh or conventional network and enough sensors are placed throughout the system to be able to resolve and monitor the full system of piping.

Along with aforementioned sensors or sensor arrays, active elements such as controlled sources of acoustic signals can also be deployed throughout the network in accordance with an embodiment of the present invention. These active elements can use encoded audio pinging or other audio signal of known shape and/or strength to communicate information inside the pipe network. Furthermore, these active elements can be used in mapping pipe network topology acting as “beacons” of known characteristics. The speed of sound inside the water and the distance from the sound source will determine the time delay at the acoustic sensor introducing the coordinates inside the pipe network.

In accordance with an embodiment of the present invention, uncontrolled sources of acoustic signal that are of interest, such as leaks or open faucets or valves, are recorded by the sensors placed throughout the network simultaneously with the controlled sources. In accordance with an embodiment of the present invention, the location of the uncontrolled source element with respect to the active elements is determined by triangulation, by combining the active element delay data with the uncontrolled source signal. The absolute and relative signal strengths can also used as part of this triangulation.

Additionally, the signal strength of the controlled active element at the sensor can be used to determine the presence or absence of fluid in the pipe. More detailed analysis of the signal strength can be used to determine the condition of the pipe. For example, a weaker signal may indicate the narrowing of the pipe due to reflection of energy from this narrowing. Further analysis can lead to a determination of pipe material properties, e.g., metal vs. plastic, by exploiting different absorption characteristics of different materials.

In accordance with an embodiment of the present invention, by constantly monitoring this network of sensors, a model for the typical flow and fluid usage in the pipes being observed can be built from the data collected. Additionally, the present invention comprises a sub-system that models pipe and flow geometry as well as the efficiency of flow and usage habits, based on the model and sensor outputs described herein. Over time, as the system is continuously monitored, data inconsistent with the established model can alert the user to the presence of an anomaly due to faults such as the existence of a leak, open faucet or valve, loss of supply or pressure, dramatic change in fluid or ambient temperature, and the like. These events can also be studied a priori so that they can be quickly identified when data surrounding a new occurrence of such an event is collected. Furthermore, because this sensor network is strategically placed throughout the piping system, it is possible, using standard correlation techniques known to those skilled in the art, to determine the specific locations responsible for the anomalous signal.

There are a number of ways to detect the presence of the analytes of interest in the materials under examination. There are reflectance modes and transmission modes of optical measurements. Examples of photometric sensing include but are not limited to spectrometric methods and can additionally comprise components for chemometric analysis.

It is appreciated that the spectrometric region from the ultraviolet to the far infrared spectral regions find utility in these applications as well as gamma, X-ray and microwave region of the available electromagnetic frequencies.

Inorganic, organic and biological contamination in potable water is a serious issue. Monitoring of the potable water supply is an important public concern. The present invention relates to the automation of water quality monitoring and alarming systems.

In accordance with an embodiment of the present invention, a cartridge system comprises that contains an array of selective growth media. These media are calorimetric such that they provide a colorimetric response when a particular biological specimen is present and active. Spectrometrically identifying the changes and quantifying the spectral response provides quantitative and qualitative analysis of biological contamination.

In accordance with an embodiment of the present invention, the system comprises a component for monitoring inorganic and/or organic substances through the calorimetric techniques. The monitoring component comprises a source filter and detector or simply an LED of limited wavelength range and a suitable detector.

In accordance with an embodiment of the present invention, the system comprises the arrangement of an array of the water quality calorimetric indicators and sensors to obtain a complete assessment of water quality.

In accordance with an embodiment of the present invention, the system comprises a SIMMS device as described in U.S. patent application Ser. No. 11/075,114, which is hereby incorporated herein by reference in its entirety. The SIMMS device installed at key locations can continuously monitor the water quality and conduct threshold contaminant alarming for commercial and/or home systems. The present system offers superior continuous baseline monitoring and recording of water quality for health, preventive maintenance, predictive failure analysis and contaminant monitoring. Additionally, in accordance with an embodiment of the present invention, the system comprises Wi-Fi networked sensor array components for complete coverage in buildings, industrial plants, municipalities and other small and/or large area systems.

In accordance with an embodiment of the present invention, the system comprises cartridges with an assortment of specific chemistries. Additionally, in accordance with an embodiment of the present invention, the system comprises a cartridge and module for biological contaminant monitoring and a separate cartridge and module for inorganic and organic chemistry monitoring. It is appreciated that varied and different sensitivities can be used to properly monitor some fluid systems with different threshold of sensitivities.

In accordance with an embodiment of the present invention, the system comprises a filter array optical sensor to “read” the color and intensity of the calorimetric reaction. A source such as a broadband lamp, a set of filters and a detector system can be used to measure spectroscopy of the calorimetric reaction or chemistry directly. The detector system can be either a single optical sensor or an array of optical sensors in any spectral region of sensitivity for the electromagnetic spectrum, including but not limited to ranges within the range from X-rays, to Millemeter length electromagnetic waves of radiation. In accordance with an embodiment of the present invention, an optical sensor that can be used in the media module can comprise a single narrow band LED or an array of narrow band LED's either of the same wavelength of operation, a combination of wavelengths, or different wavelengths but in single band.

In accordance with an embodiment of the present invention, the system comprises a component to measure resistance as a sensor for the media. Additionally, in accordance with an embodiment of the present invention, the system comprises a component that applies an electric potential to assist molecular migration. Further, in accordance with an embodiment of the present invention, the system comprises a heating component for applying heat in a biological module to assist selective culture propagation and/or in a chemical module to assist a reactive reagent.

In accordance with an embodiment of the present invention, the system comprises a tuned light system as described in U.S. Pat. No. 6,859,275, which is incorporated herein by reference in its entirety. The tuned light can be used to assist reactions for indirect and direct measures and calorimetric activity.

Various colorimetric indicator embodiments described herein can take advantage of the abundance and availability of certain LED's spectral ranges. However, it is noted that colorimetric reactions are not always required to qualitatively and quantitatively measure constituents in the sample matrix. In accordance with an embodiment of the present invention, the method comprises optical detector methodology that employs spectrometry to spectrometrically identify many compounds, including the identification of compounds in states that can be found on thin layer chromatography (TLC) plates. In accordance with an embodiment of the present invention, the method comprises a calorimetric or non-colorimetric monitoring of TLC plates. As will be readily seen by one skill in the art, the discussions herein about the interchange of spectrometric techniques for calorimetric reactions applies to many of the embodiments described herein.

FIG. 3 shows a view from above of a reagent test strip. Blocks of test reagent (b) are deposited upon a backing strip (a). Typically, each of the reagent blocks is different, and each block tests for a different chemical constituent in the fluid to be tested.

FIG. 4 shows a side view of a reagent test strip. Blocks of test reagent (b) are deposited upon a backing strip (a). Typically, each of the reagent blocks is different, and each block tests for a different chemical constituent in the fluid to be tested.

In another aspect, the present invention provides a rapid and simple methodology for measuring chemical constituents of a fluid. The present invention has the advantage that it can make multiple chemical determinations in a single application, thereby improving the throughput of the process, and that it only requires low-cost reagent strips as consumables. In accordance with an embodiment of the present invention, the system and device can be made small, portable, rugged and self-contained except for a power supply. Additionally, the device and system is sealed against damage from fluid immersion. The present invention comprises a test-strip and a test-strip reader. The test-strip reader holds the test-strip and the fluid sample. The test-strip reader either comprises or is accessible to one or more calorimetric sensors. The sensors transmit their information to a computational unit which interprets the measurements and calculates the presence of, or concentrations of chemicals in the sample.

In accordance with an embodiment of the present invention, the test-strip is configured similar to the conventional test-strips as described in FIGS. 3 and 4. It is appreciated that the reagent blocks on the test-strip are known to the test-strip reader. Depending on the colorimetric sensor which is used with the device of the present invention, the test strip upon which the reagent blocks are deposited can have special properties. For example, if a transflectance sensor is used, then the strip should be reflective at the required optical wavelengths. Alternatively, if a transmission sensor is used, then the strip should be transparent, or even have a hole in the strip to pass the optical wavelengths or allow the sample to disperse around the colorimetric sample chamber.

In accordance with an embodiment of the present invention, the test-strip reader brings together the test-strip, the fluid, and one or more colorimetric sensors sensitive to the expected colorimetric change. The test strip reader is designed such that the test-strip is easily replaceable, the fluid can be readily introduced into the system, and a multitude of repetitive tests can be easily conducted on a multitude of samples. The test-strip reader is designed such that when the sample is introduced into the system, portions of the sample are guided to each of the reagent blocks on the test-strip. In accordance with an embodiment of the present invention, the test-strip reader is designed such that the reagent blocks, now immersed in fluid sample, are held in position to be read by the calorimetric sensors which are either an organic part of the test-strip reader, or otherwise attached to the test strip reader.

In accordance with an embodiment of the present invention, the test strip comprises more than one reagent block. A method of the present invention for observing the reagent block with the colorimetric sensor, include but is not limited to the use of one colorimetric sensor for each reagent block, or the use of more reagent blocks than sensors, wherein at least one sensor is disposed to observe multiple reagent blocks.

In accordance with an embodiment of the present invention, the system and method utilizing one detector per reagent comprises the elements shown in FIG. 5. In FIG. 5, the test-strip reader is configured to contain independent wells into which the reagent blocks on the test-strip fit. The interval between wells is the same as the interval between reagent blocks on the test-strip. The test-strip reader is also configured so that when fluid is introduced into the test-strip reader portions of the fluid flow into each of the wells. The test-strip reader can be immersed into a large body of fluid, or can comprise channels if only a small amount of fluid is available. Alternatively, a portion of fluid can wash over the test-strip reader from one end to the other, and naturally flowing into the wells.

Associated with each of the wells is a calorimetric sensor. In FIG. 5, the sensor is shown as embedded in the test-strip reader below each of the wells. This geometry can be appropriate for a transflectance colorimetric measurement, although other types of colorimetric sensing are also possible with different geometries.

FIG. 5 shows a side view of a test-strip reader in accordance with an embodiment of the present invention. Test strip (a) with attached reagent blocks (b) is immersed in a fluid filled container (c). The container (c) has fluid-filled wells into which the reagent blocks fit. Each well is monitored by calorimetric sensors (d).

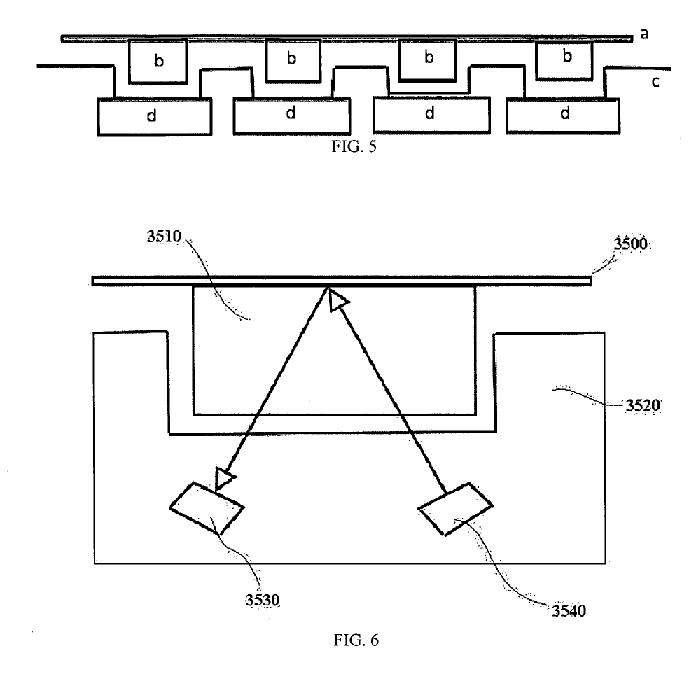

FIG. 6 shows the colorimetric sensor operating in a transflectance mode in accordance with an embodiment of the present invention. The colorimetric sensor is embedded in the body of the test-strip reader. Light emitted by the embedded light source makes two passages through the sample with colorimetric reagent, and the reagent block before being detected by the embedded light receiver. The colored light is emitted by a narrow-band source 3540, passes through the reagent block 3510 and is reflected by the reflective test-strip backing 3500. The light passes again through the reagent block 3510 and is sensed or detected by an optical sensor 3530. The body of the test-strip reader 3520 is transparent to the relevant wavelengths of light.

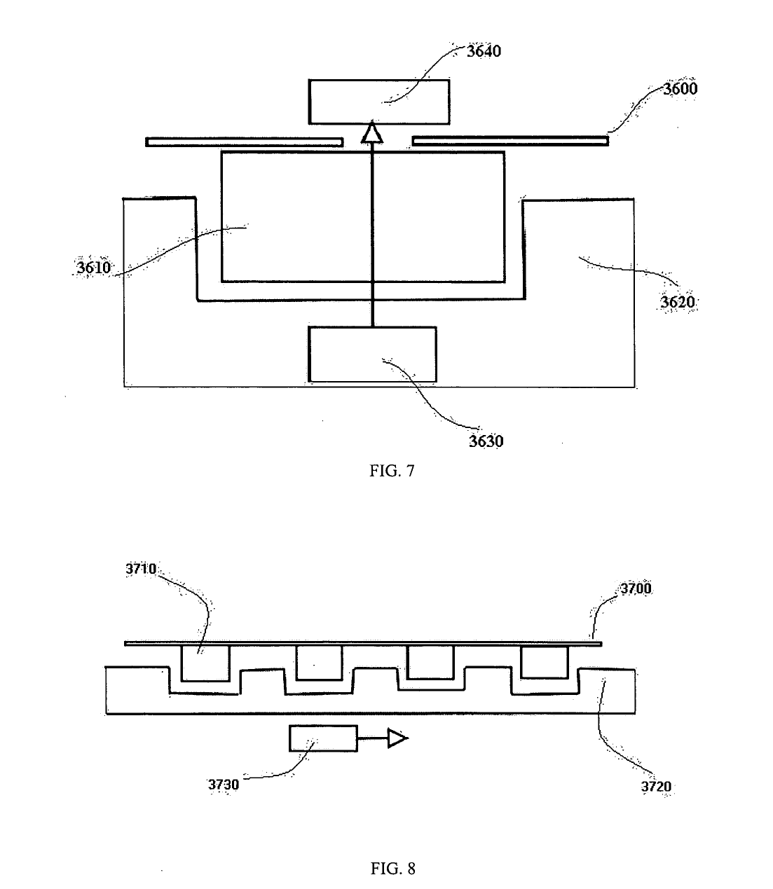

FIG. 7 shows the colorimetric sensor operating in a transmission mode in accordance with an embodiment of the present invention. The test-strip reader holds the test-strip between a light source and a light sensor. The test-strip backing is operable to pass the optical wavelength being used, either being effectively transparent or having a physical hole in the backing at the appropriate location. The colored light is emitted by a narrow-band source 3630, passes through the reagent block 3610, passes through a hole in the test-strip backing 3600, and is sensed or detected by an optical sensor 3640. The body of the test strip reader 3620 is transparent to the relevant wavelengths of light.

An advantage of the embodiments illustrated in FIGS. 5, 6 and 7 is that the colorimetric sensor can be made compact, rugged, sealed against immersion, and possessing no moving parts. Its external interface can be supplied via an attached cable which supplies power for the light sources and brings out data from the colorimetric sensors. The unit can be immersed in a fluid to be measured by its cable or if desired, by a rigid tether. This can be advantageous for non-laboratory applications.

FIG. 8 illustrates a single calorimetric sensor which is multiplexed among the reagent blocks in accordance with an embodiment of the present invention. The colorimetric sensor is mechanically scanned from one reagent block to another, and this may be preferable if the colorimetric sensor is expensive. However, it may be more difficult to make the scanning apparatus rugged and immersion-safe than the non-scanning apparatus described herein. In this manner, the present invention provides the advantage of allowing the system implementer to choose a point along the cost-performance tradeoff in a way that is not available in the prior art. This tradeoff can be accomplished by having N detectors multiplex the reading of M reagent blocks. By selecting the number N, the tradeoff can be realized.

In FIG. 8, the test strip 3700 with the attached reagent blocks 3710 fits into wells in a test-strip holder 3720. Fluid is introduced into the test-strip and fills the wells in the test-strip holder 3720. A calorimetric sensor 3730 is scanned from one reagent block to another, making calorimetric measurements of each reagent block.

There are a number of possibilities for the colorimetric sensor. In accordance with an embodiment of the present invention, the colorimetric sensor can operate on a single narrow band of wavelengths or can comprise an off-the-shelf three color sensor. In accordance with an embodiment of the present invention, the sensor comprises many spectral bands, such as those disclosed in U.S. Pat. Nos. 6,392,748 and 6,859,275, each of which is incorporated herein by reference in its entirety.

In accordance with an embodiment of the present invention, the system and device comprises a computational unit for taking measurements from the calorimetric sensors and making a determination of the concentration. The output of the colorimetric sensors depends on a variety of factors, including the thickness, composition, and diffusion coefficient of the reagent block, the concentration of the relevant chemicals in the fluid, the temperature of the apparatus and the fluids, and the amount of time that the test-strip has been in contact with the fluid. The computational unit performs computations which remove the effect of these other factors and produce a determination of the concentration of the relevant chemical in the fluid, as disclosed herein.

The output from the calorimetric sensor is a function of time. In the moment before the reagent block is first placed in contact with the fluid, the reagent block is in an unreacted state. Once the fluid is placed in contact with the reagent block, the fluid begins to diffuse into the interior of the reagent block. The diffusion process is not instantaneous, but occurs over a perceptible interval. The speed with which the chemicals diffuse into the reagent block depends upon the shape and size of the reagent block and the diffusion coefficient of the chemical in the reagent block material. Once the chemicals in the fluid have come into contact with the reagent, chemical reactions occur between the chemicals in the fluid and the reagent in the reagent block. The chemical reactions are also not instantaneous. The colorimetric sensor measures attenuation in light of a certain wavelength which has passed through the reagent block and been affected by the by-products of the reactions between the reagent and the chemicals in the fluid. The rate of diffusion and the rate of chemical reactions are dependent upon temperature.

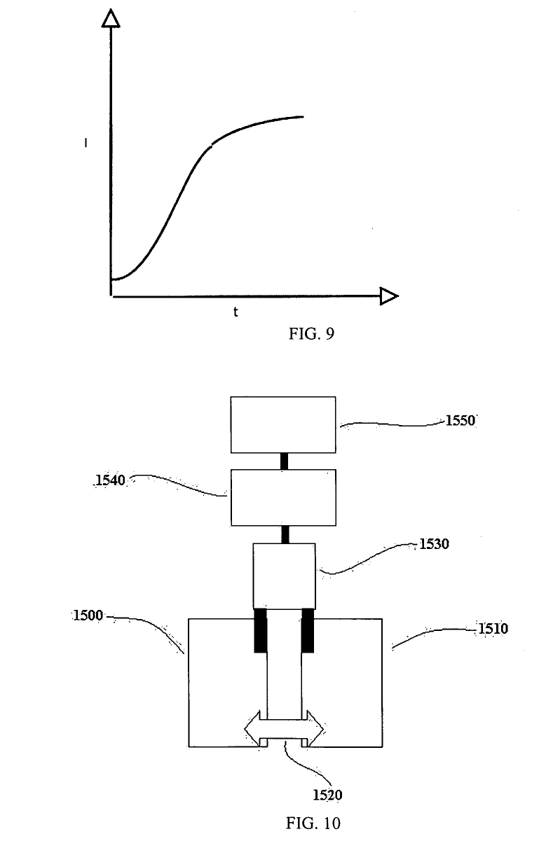

FIG. 9 illustrates a typical time evolution of the signal intensity from a colorimetric sensor in accordance with an embodiment of the present invention. The signal intensity evolves in time until all the time-varying processes (diffusion, chemical reactions) have reached a steady state, at which time the signal asymptotically converges to a constant. A common problem in various colorimetric measurement schemes is that it is not easily apparent when the steady state has been reached. This leads to the user waiting for unnecessarily long time before taking the measured intensity as representative of the steady state.

Typically, analysis must wait until this asymptotic value is closely approached before making a determination of chemical concentrations. In accordance with an embodiment of the present invention, the method comprises the step of resolving the converged intensity from earlier readings, thereby enabling faster readout. The reaction intensity curve (like FIG. 9) will be a function of temperature, concentration, and time if the size, shape, and materials of the reagent block are held unchanging. Therefore, it is possible to produce a number of curves in the laboratory which fully characterize the possible measured intensity curves. These curves, or parametric representations thereof, can be stored in the computational unit and compared to the actual measured reaction intensity curve. Furthermore, it is not necessary to compare to the entire reaction intensity curve, but comparison can be made to only the early portion of the measured intensity curve. The comparison determines which interpolation of the stored intensity curves is similar to the measured intensity curve. The comparison is accomplished through a non-linear least squares algorithm or similar algorithms. This allows the reliable determination of the final steady state of the intensity curve using only the early time part of the curve. This improves the throughput of the measurement operation without sacrificing accuracy. It may even potentially improve the accuracy of each measurement.

Transmission of the data from the calorimetric sensors to the computational unit may be made via cable or wirelessly, which is advantageous in some situations. Also the analysis from the computational unit may be printed for an end-user, or transmitted to other computational units for higher level analysis. Again, any transmissions can take place via cable or wirelessly, which is advantageous in some situations.

In accordance with an embodiment of the present invention, the system and method monitors leaks in pools and other containers. FIG. 10 is a block diagram illustrating such system comprising a sample cell 1500, reference cell 1510 and means 1520 for thermally coupling them. A pressure transducer or other sensor 1530 monitors the fluid in each cell and makes either two measurements or a single measurement that reflects a comparison of the two cells (e.g. a pressure differential). The data is either digital, or is digitized by an analog to digital converter (ADC), and is processed by a data acquisition and signal processing unit 1540. The derived information and device controls are presented by the user interface 1550.

In accordance with an exemplary embodiment of the present invention, an ⅛′ decrease constitutes about 13 ADC counts. If the device measured three sequential periods of 160 minutes each, and each period had at least one ADC count less than the previous period, this would indicate a leak. Three 160 minute periods is 8 hours. If any period showed an increased (of even one) ADC count, then the process can restart. The system can show an alert (red light) after three sequential periods of decline. The alert can be automatically reset by a change in the measured conditions. This change could be rain, or user removal of the device, or other event that can act as a reset mechanism. In accordance with an exemplary embodiment of the present invention, the level for a given period can be calculated by taking the average of 8 measurements that are 200 ms apart. The value is then stored and compared to the next measurement in 160 minutes. If the new measurement is higher (in pressure) then the alarm state is reset to 0. If the new measurement is less than the previous, the alarm state is incremented. If the alarm state is 3 or more, then the system determines that a leak has been detected and an alert is shown. An alert is shown by turning on a LED for 500 ms and then waiting 6 seconds. This conserves battery energy while showing a leak alert and continuing to measure the pool level. The specific numbers discussed herein are meant to represent parameters of the system and of course can be replaced by any other appropriate values.

One example of instruction for use of a leak detector for a residential swimming pool is a follows. The associated consumer operated leak indicator is a device that can be used easily by consumers to check for leaks in pools, spas or other fluid containing vessels. The device simply attaches to the wall of the pool or spa at the waterline. Note that this is not restricted to residential swimming pools:
1.) All pumps and valves are turned off and the pool must not be used for the duration of the test. Typically an overnight test greater than four hours is recommended.
2.) The user attaches the mount system so that the level of the device is at the water line.
3.) The user scoops up a sample of the water into the device using the integrated scoop and fills it to the range indicated by two lines scribed on the clear scoop of the container.
4.) The user observes that the green light begins to blink indicating a test is underway. An amber blinking light indicates that there is an error and the device needs to be reset as described in step 7. The user should then repeat the process from step 3 on.
5.) The user leaves the device for at least four hours to do a full leak test.
6.) Upon returning four hours later or overnight the user observes the color of the LED light.
   a. If a leak is indicated by a red color LED light then the user can either repeat the test and confirm or call for professional assistance to further identify and/or repair the leak.
   b. If a green light is indicated the user can rest assured that there is no leak.
   c. If an amber light is indicated then there has been an interruption in the test such as rainfall, pool use, or pool tampering. The user should then reset the device and repeat the test.
7.) If the user needs to reset the device.
   a. Remove the device from the pool or spa and empty the reference cell container by pouring the water out.
   b. Wait 1 minute for the device to reset indicated by a series of alternating green and red flashes.
   c. Refill and reset the device into the water as per above instructions.

The pool leak detector in accordance with an embodiment of the present invention provides an inexpensive and reliable way to monitor pools and spas for leaks throughout the year, thereby conserving water and avoiding unnecessary water and chemical use and costs. For example, the pool leak detector of the present invention can detect level changes as small as 3/16^{th} of an inch. The device provides for a simple fill and set operation. The device can be made with a durable plastic molded case with integrated scoop. The tri-function LED leak status indicator light provides for simple non-expert operation, wherein the no-fault operation automatically detects a faulty leak test. In accordance with an embodiment of the present invention, the system comprises automatic evaporative loss compensation, and automatic barometric pressure compensation. Preferably, it is small and lightweight (for example, ¾ inches by 2 inches by 7 inches). The device consumes a small amount of power, and many tests are possible from a single 9V battery. The device works in any size pool or spa.

While aspects of the present invention are described herein in terms of the monitoring of swimming pools, many other fluid carrying tanks, containers and the like can be measured with the present invention.

In accordance with an embodiment of the present invention, the system and device can additionally comprise chemistry measuring components, to measure such things as chlorine, sanitizer, pH and Turbidity.

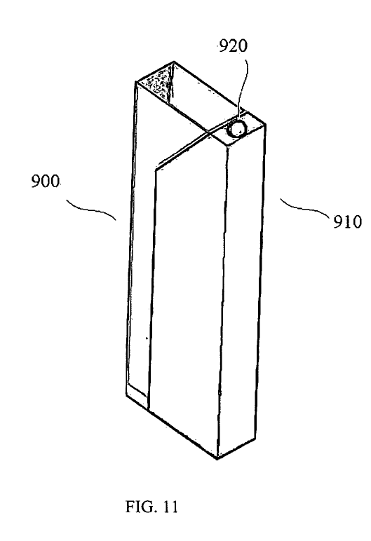

In accordance with an embodiment of the present invention, sensitivity of the measurement of the fluid level in a container is increased when using a differential transducer when the reference and sample chambers are similar in dimension. FIG. 11 shows a pool leak detection system and device in accordance with an embodiment of the present invention, which is comprised of two chambers 900 and 910, a sensor system (not shown), a processing system (not shown), and an LED 920. The chamber on the right 910 (i.e., the sample chamber 910) is filled through a baffled inlet at the bottom. As the pool level changes, the sample chamber level will also change. The chamber on the left, the reference cell chamber 900, is roughly the same dimension as the sample chamber 910 and has no inlet to the pool. It is filled at the beginning of the test. The attribute of similar dimensions dictates similar resonant response function to oscillations in the fluids caused by various environmental noises and stimuli. This creates a nulling effect in the data so that only the pressure differential of the change in the sample chamber fluid level is measured and noises in the measure are cancelled out due to the matching dimensions on the sample and reference cell. An improved leak measurement device is now possible.

In accordance with an embodiment of the present invention, a Micro-Electro-Mechanical System (MEMS) differential pressure sensor measures the difference between ambient (atmospheric) pressure above the water and pressure inside the tube caused by the volume of pool water compressing the air in the tube. By measuring this frequently and analyzing the rate of change of the pressure difference, the present invention can infer the amount of change in the volume of water in the pool. By modeling live regular usage (evaporation) conditions, it is then possible to detect when there is abnormal water loss, i.e. a leak. In accordance with an embodiment of the present invention, the evaporation measurement and model comprises additional sensors and information including but not limited to some or all of the following: water temperature vs. time, air temperature vs. time, relative humidity, the chemical composition of the pool water over time, wind speed over time, the dew point, and the surface area of the pool. In accordance with an embodiment of the present invention, the system and device additionally comprises a float to sit on surface of water and mounted with temperature sensors. Temperature in the water near the surface is measured, as well as ambient (out of water) temperature which is measured on top of the float, or on the head of main unit. A humidity sensor can be also located on the head of the unit.

In accordance with an embodiment of the present invention, the device conserves power by being mostly inactive via an intelligent “sleep mode” as described herein. In such a mode, the device only powers itself up when making a measurement.

In accordance with an embodiment of the present invention, the device can additionally comprises components, methods and/or systems to augment the evaporation measurement and model described herein, by the determination, measurement, modeling and/or input of the pool refilling, splashing and dynamic motion in pool

In accordance with an embodiment of the present invention, the system, device and method comprises monitoring the rate of change (1st derivative) of the water level over time. Positive changes and shocks (i.e. when pool is refilled, or object falls into pool, etc.), can be treated differently from regular (smoothly varying) decreases in level. In particular, in a mode of operation that is simply looking for water loss, the positive changes and shocks can be ignored. Also, in other modes, the present invention can use the increases to model pool filling, refilling and rain. Shocks can be used to model pool activity and use. In accordance with an embodiment of the present invention, the system and device can additionally comprise a pool alarm system, wherein shocks and activity in the pool, as described herein, causes an alarm condition. The activity alarm system of the present invention can further comprise standard alarm system components, including but not limited to a monitoring circuit, an override circuit, and a reset. Techniques including but not limited to statistical classification and regression techniques, can be used to discriminate between dynamics and motion including but not limited to thermal changes, wind, debris falling in the pool vs. pool use, animals entering or falling into the pool, the difference between swimming and distress, the difference between adults, children and babies, and the like.

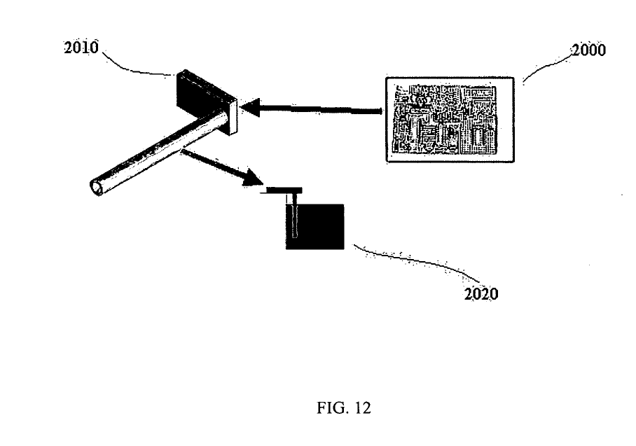

FIG. 12 illustrates an embodiment of the present invention showing a device 2010 comprising an exemplary electronics module 2000 in a deployment mode 2020. The present device is a more robust, accurate, simple to use, and less expensive leak detection system. Preferably, the device has consumer no-fault operation and can be used for commercial pool monitoring. In accordance an embodiment of the present invention, the device is low-cost, battery operated, has onboard sensors to compensate for evaporative losses, allows for a product line including automated pH balance and chlorine detection, as well as other sensors, and wireless control of other pool accessories and equipment.

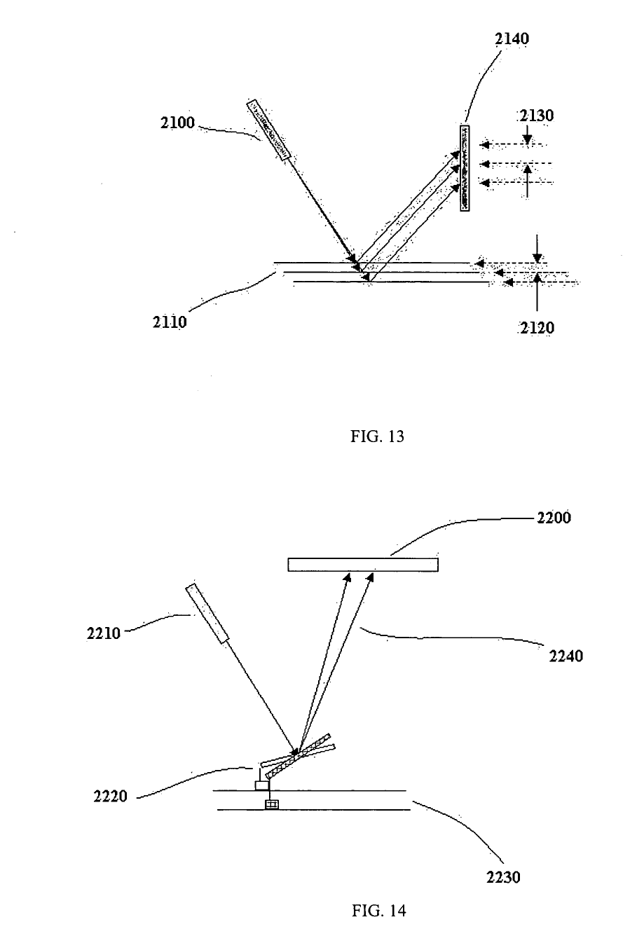

FIG. 13 shows how a laser or other light source 2100 can be used to measure water level changes by reflection at an index of refraction interface or surface in accordance with an embodiment of the present invention. In one exemplary configuration, the laser source 2100 approaches the surface of a material or fluid in a container at an angle a such that as the level changes, the reflected pattern is translated in space. The reflected pattern can be a bright spot of a laser beam impinging upon a frosted glass sight window 2140 situated such that an operator can view or a device can record this change over time. The magnitude of the movement 2130 with respect to the level change 2120 of the material (or fluid) under observation depends on the angle α, index of refraction, and angle at which the window 2140 is placed. Three possible exemplary levels of material 2110 are shown in FIG. 13.

FIG. 14 shows how a system can be arranged to measure the level change of a material where magnification of the indication of the level change is desired in accordance with an embodiment of the present invention. A pivoting mirror or reflective surface that is attached to a float 2220 on the surface of the material such as water in a container causes the mirror to rotate by an angle a about an axis such that the impinging light energy from the source 2210 is reflected to a different location on a sight glass or detector 2200 placed some distance away. The movement of the source beam 2210 at the plane of detection is amplified by the 2α condition of reflection and the ratio of the difference between the mirror pivot arm and the distance from the point of reflection to the point of detection. The magnitude of movement can be detected by a linear or two dimensional detector, a linear or two dimensional focal plane of detectors, or by a sight glass scale 2200 and/or observer. The 2α amplification 2240 is illustrated in FIG. 14 by showing the float 2220 and pivoting mirror in two rotated positions corresponding to two different possible water levels 2230.

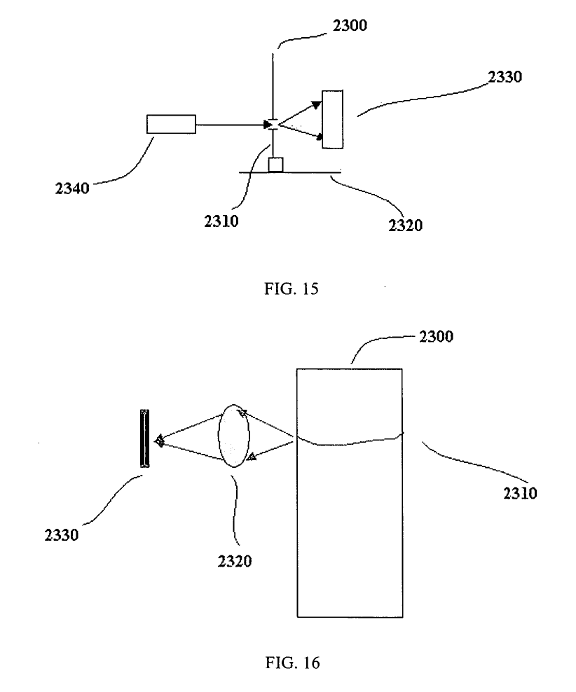

FIG. 15 shows a diffractive method utilizing slit and coherent source 2340 can be used to ascertain the magnitude of level change or movement in accordance with an embodiment of the present invention. The diffracted pattern changes with the size of the slit or aperture such that if the aperture size is dependent upon the level 2320 of the material, the present invention can determine the magnitude of a level change by the observed magnitude of the diffracted pattern change via a linear or two dimensional detector or sight glass 2330. A slit aperture is constructed so that the top portion 2300 is fixed, while the bottom portion 2310 is not fixed but allowed to move vertically and coupled to a float in the material. Hence the movable bottom portion creates an aperture whose size will track the level of the material. The coupling and float system can incorporate classical mechanical gearing techniques known to those skilled in the art in order to amplify or de-amplify the level change.

FIG. 16 shows how direct imaging using magnifying or imaging optics and digital camera can be used in accordance with an embodiment of the present invention to detect a level change. The imaging optical systems can be used to directly measure the level change in a container 2400. The optical imaging system 2420 can directly measure the magnitude of the movement of the image of the surface or meniscus 2410 of the material by determining the amount of shift of the image on the detector array 2430. The magnitude of movement can be detected by a linear or two dimensional focal plane of detectors or by a sight glass scale and/or observer. It is appreciated that various conventional optical systems can be used to magnify the level change for the observer.

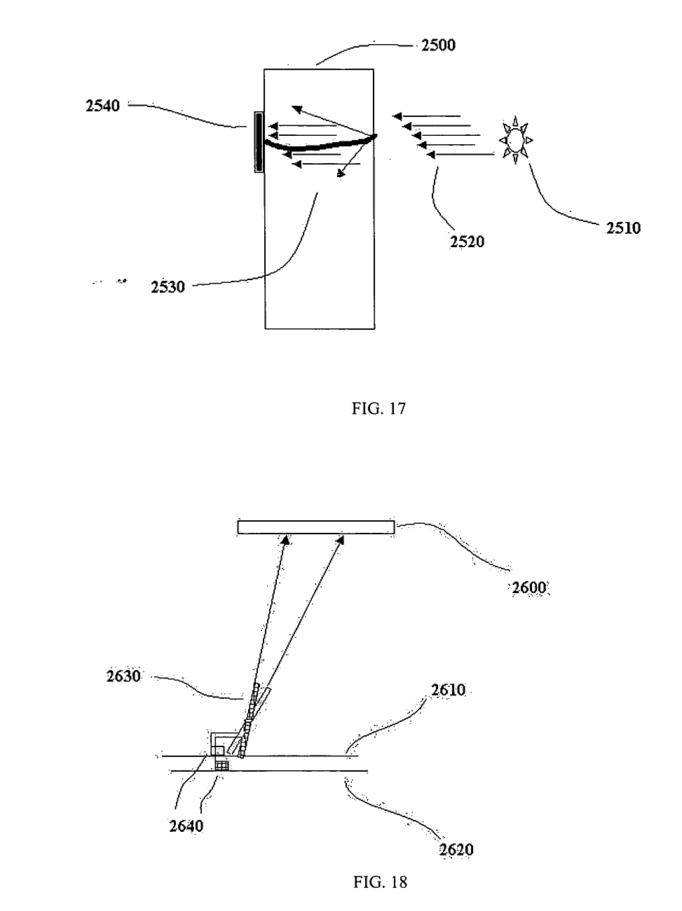

FIG. 17 shows how broadband light source 2510 and a linear or 2D detector 2540 can be used in accordance with an embodiment of the present invention to detect level changes by analyzing the pattern movement on the detector 2540 with time. The radiation 2520 from the source 2510 impinges on the side of an at least partially transparent container of material 2500 and passes through a portion of said container which includes the surface or meniscus of the contained material as indicated by the dark line drawn in the container 2530, thereby producing a fixed pattern of scattered radiation that reaches the detector 2540. The movement of such pattern is detectable by an observer and a device such that level changes can be directly measured. The magnitude of movement can be detected and measured by a linear detector or a two dimensional focal plane of detectors or by a sight glass scale and/or observer. It is appreciated that optics can be used to magnify this change for the observer.

FIG. 18 illustrates the direct movement of the source beam caused by material level change in accordance with an embodiment of the present invention. The lines 2610 and 2620 represent the two sample material levels in FIG. 18. The beam source, mounted on a center-pivot, is connected to a float on the surface of the liquid. In the exemplary embodiment as shown in FIG. 18, the system is drawn in two states: 2640 shows the float 2640 is shown in two positions corresponding to the two material levels 2610 and 2620, and the beam source 2630 is shown at the two corresponding angles. A detector or sight glass 2600, possibly graduated, allows the user to measure the change in material level by observing the motion of the spot from the impinging source beam 2630. The two beam positions from the beam source 263 reach the detector 2600 in two distinct and observable positions. In this example, the float 2640 has a rigid mount which connects to a pivot point on the center-pivoted beam source 2630. It is clear to those skilled in the art that these mechanical couplings could be designed in many ways with various rigid or flexible couplings, pivots, and pulley systems.

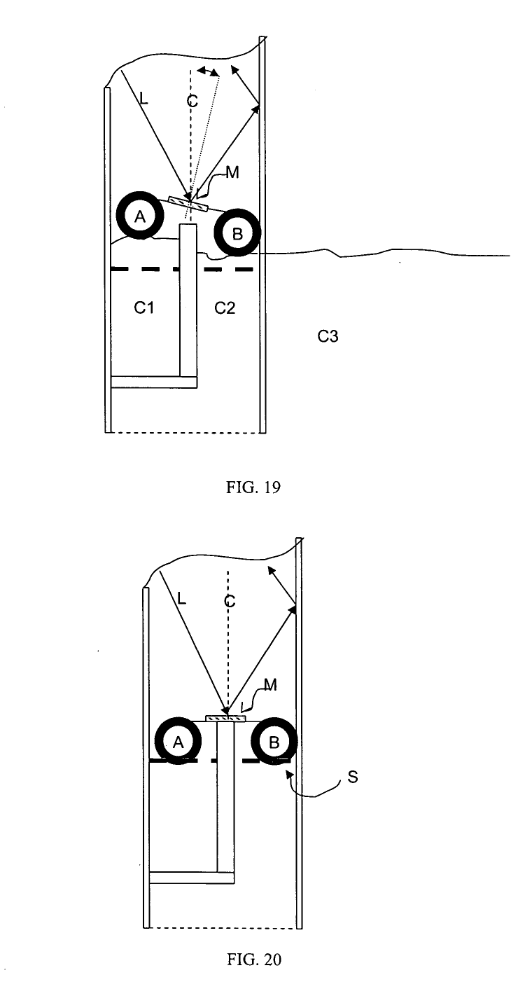

The present invention allows rapid assessment of small level changes in large containers. In accordance with an exemplary application of the present invention, the device, system and method can be used to measure leaks in swimming pools for rapid troubleshooting of leak problems. A mirror-hinge-float assembly can be used to detect leaks or level changes rapidly. An exemplary application of the present invention for measuring/detecting leaks in a swimming pool is shown in FIG. 19. A laser or other source of light energy or beam of light L is fixed and made to impinge upon a mirror M such that a rotation of mirror M would result in a change of position of the reflected beam. The encounter with a curved or flat mirror M rotated through an angle would result in an angle change to the reflected beam at twice that angle. The Length Z of travel of this reflected beam multiplies the change in position Δ of the beam at the length Z of travel. The level change of the fluid Y as referenced by cell C1 has an effect on the position terminus of the beam of light at detector D as shown in FIG. 19. The benefit of having a cell C1 is to compensate for environmental effects such as that of evaporation. The fluid or material in C3 referenced and equal in composition to the fluids in C2 will evaporate at the same rate as C1 such that evaporative effects are compensated for in the resultant position of the beam at detector D. Detector D is a linear array of detector elements or ruled sight glass such that the position of the beam on this detector array or sight glass is indicative of the level difference at some time between C1 and C2. Note that C2 and C3 are the same level at all times. In accordance with an embodiment of the present invention, the device is self calibrating as shown by placement of a screen S in FIG. 20. An empty calibration position of mirror float assembly is shown in FIG. 20 such that the mirror is normal or at a fixed position when no material is present or under test. This allows the system to be easily and rapidly calibrated before use, in accordance with an embodiment of the present invention.

The use of the present device in accordance with an embodiment of the present invention is to secure the device to the side of a container or pool such that an adjustment by way of a wheel gear assembly can lower the device into the fluid or water such that the cell C1 is filled. The device is then adjusted such that the mirror float assembly is close to normal position as indicated by the placement of the laser beam onto the center of the detector array or sight glass in the start area of the detector. It is appreciated that the exact placement of the terminus of the beam is not important in every aspect, but in some embodiments it is disposed to impinge upon the approximate center of the target detector D in the alignment state as described herein. The magnitude of the movement of the beam over time is dictated by the length Z and the level change of fluids. The system can be designed to increase the magnitude of the sweep of the beam of light and used to measure the level change of the fluid very precisely in a short amount of time.

In accordance with an exemplary embodiment of the present invention, the device uses a laser diode to reflect off of a standard flat mirror and impinge a predetermined number of inches (for example, 24 inches) away on a liner detector array with a predetermined number of detectors per inch (dpi), (e.g., 400 dpi). With these exemplary numbers, the present invention is capable of detecting a change in position of over 12 detector elements in 10 minutes when a 20 ft by 40 ft pool is leaking 3/16^{th} of an inch over a 24 hour period. This detection is 4 times the Nyquist sampling rate for such a level of sensitivity.

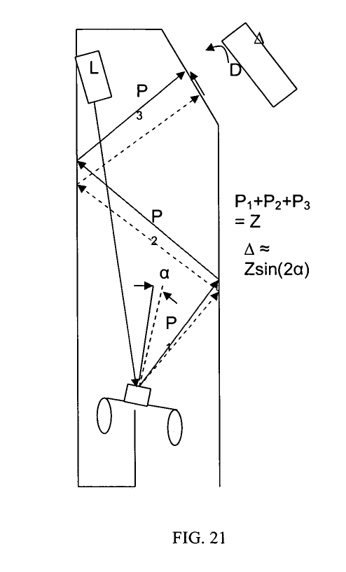

FIG. 21 shows how a displacement on a sensor corresponds with change in fluid level in accordance with an embodiment of the present invention.

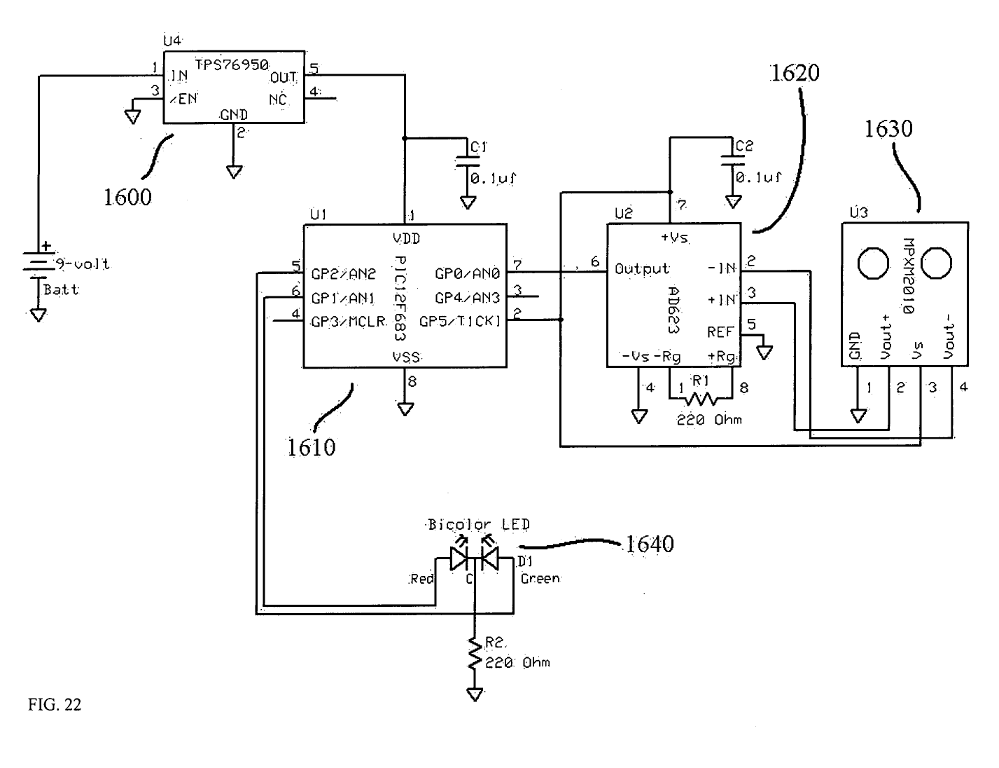

FIG. 22 shows a schematic for an exemplary prototype of a pool leak detector in accordance with an embodiment of the present invention. The circuit shown in FIG. 22 illustrates a basic design comprising only five integrated circuits (ICs): a Texas Instruments TPS76950 voltage regulator 1600, microchip PIC12F683 microcontroller 1610, an Analog Devices AD623 amplifier 1620, a Freescale MPXM2010 MEMS pressure transducer 1630, and typical bi-color LED 1640.

While the foregoing has described and illustrated aspects of various embodiments of the present invention, those skilled in the art will recognize that alternative components and techniques, and/or combinations and permutations of the described components and techniques, can be substituted for, or added to, the embodiments described herein. It is intended, therefore, that the present invention not be defined by the specific embodiments described herein, but rather by the appended claims, which are intended to be construed in accordance with the well-settled principles of claim construction, including that: each claim should be given its broadest reasonable interpretation consistent with the specification; limitations should not be read from the specification or drawings into the claims; words in a claim should be given their plain, ordinary, and generic meaning, unless it is readily apparent from the specification that an unusual meaning was intended; an absence of the specific words “means for” connotes applicants' intent not to invoke 35 U.S.C. §112 (6) in construing the limitation; where the phrase “means for” precedes a data processing or manipulation “function,” it is intended that the resulting means-plus-function element be construed to cover any, and all, computer implementation(s) of the recited “function”; a claim that contains more than one computer-implemented means-plus-function element should not be construed to require that each means-plus-function element must be a structurally distinct entity (such as a particular piece of hardware or block of code); rather, such claim should be construed merely to require that the overall combination of hardware/firmware/software which implements the invention must, as a whole, implement at least the function(s) called for by the claim's means-plus-function element(s).

We claim:

1. Apparatus for detecting leaks in a fluid vessel, comprising a pressure sensor for measuring a pressure difference between a reference cell and a sample cell, said sample cell being open to said fluid vessel; and wherein said apparatus detects a leak in said fluid vessel when the pressure difference exceeds a predetermined threshold.

2. The apparatus of claim 1, wherein said fluid vessel is a swimming pool.

3. The apparatus of claim 2, further comprising a device for mounting said apparatus on a side of said pool.

4. The apparatus of claim 1, wherein said fluid vessel is a oil storage tank.

5. A method of detecting leaks in a fluid vessel, comprising the steps of measuring a pressure difference between a reference cell and a sample cell, said sample cell being open to said fluid vessel; and detecting a leak in said fluid vessel when the pressure difference exceeds a predetermined threshold.

6. The method of claim 5, wherein the step of detecting comprises the step of detecting a leak in a swimming pool when the pressure difference exceeds a predetermined threshold.

7. Apparatus for detecting leaks in a fluid vessel, comprising:
a float;
a mirror mounted on said float, said mirror tilting based on the position of said float; and
an array of detector elements for receiving light energy reflected from said mirror; and
wherein a leak is determined based on one or more detector elements that are impinged by said light energy reflected from said mirror.
